(19) United States
(12) Patent Application Publication
(10) Pub. No.: US 2023/0038389 A1
(43) Pub. Date: Feb. 9, 2023

(54) SYSTEMS AND METHODS FOR KINK DETECTION IN A CANNULA

(71) Applicant: Cercacor Laboratories, Inc., Irvine, CA (US)

(72) Inventor: Hung The Vo, Fountain Valley, CA (US)

(21) Appl. No.: 17/817,602

(22) Filed: Aug. 4, 2022

Related U.S. Application Data
(60) Provisional application No. 63/229,403, filed on Aug. 4, 2021.

(51) Int. Cl. G01L 9/00

(52) U.S. Cl. CPC G01L 9/0072

(57) ABSTRACT

Systems and methods relate to a kink or obstruction detection system for use in a minimally invasive implant, such as a disease management device. The kink or obstruction detection system can detect changes in pressure in the fluid flow path of a fluid being delivered to a patient. The kink or obstruction detection system can be included in a device between a pump and the cannula.

### FIELD OF THE DISCLOSURE

The general field of this disclosure is glucose sensing and disease management systems.

### BACKGROUND

Diabetes is a chronic disease that impacts many individuals, both adults and children. The management of diabetes may include the measurement of glucose within the interstitial space including blood and/or interstitial fluid of a patient and administration of insulin to the patient. A closed loop insulin administration system includes both a sensor to take glucose measurements from the interstitial space including blood and/or interstitial fluid of the patient and an insulin administration device which administers insulin to the patient based on the glucose measurements. Closed loop insulin administration systems allow individuals impacted by diabetes to go about daily life with much less worry about their insulin or glucose levels which can vastly improve a diabetic's quality of life.

### SUMMARY

Various aspects of systems, methods and devices within the scope of the appended claims each have several aspects, no single one of which is solely responsible for the desirable attributes described herein. Without limiting the scope of the appended claims, some prominent features are described herein.

In some aspects, a disease management device can comprise: a medical delivery pump configured to deliver a medication from a medication pouch to a patient via a cannula implanted in a patient; and a kink detection system configured to measure an obstruction in the cannula, the kink detection system comprising: a fluid receiving portion configured to receive fluid from the medical delivery pump; a diaphragm coupled to the fluid receiving portion, the diaphragm configured to deform in correspondence with a change in pressure in the fluid received; a sensor configured to measure an extent of deformation of the diaphragm; one or more hardware processors configured to: determine a pressure in the fluid based on the extent of deformation; determine if a change in the pressure in the fluid passes a threshold change; and output an alert to the patient if the change in pressure passes the threshold change; and a fluid outlet configured to deliver the fluid to the patient.

In some aspects, the sensor can comprise a capacitive sensor.

In some aspects, the sensor comprises a conductive sensor.

In some aspects, the capacitive sensor is located with in a capacitive sensing circuit, the capacitive sensing circuit comprising: a bias resistor; a touchpad; a capacitor; and a controller.

In some aspects, an electrical capacitance of the touchpad is configured to correspond to movement of the diaphragm.

In some aspects, wherein the conductive sensor is located within a conductive sensing circuit, the conductive sensing circuit comprising: a bias resistor; a conductive strip; and a controller.

In some aspects, the conductive strip is configured to stretch as the pressure in the fluid is increased.

In some aspects, the one or more hardware processors are further configured to communicate with peripheral devices via bluetooth.

In some aspects, a detection system configured to detect an obstruction within a fluid flow path, the detection system comprising: a fluid receiving portion configured to receive fluid from a delivery pump; a diaphragm coupled to the fluid receiving portion, the diaphragm configured to deform in correspondence with a change in pressure in the fluid received; a sensor configured to measure an extent of deformation of the diaphragm; and one or more hardware processors configured to: determine a pressure in the fluid based on the extent of deformation; determine if a change in the pressure in the fluid passes a predetermined pressure threshold; and output an alert to a user if the change in pressure passes the predetermined pressure threshold.

In some aspects, the sensor is a capacitive sensor.

In some aspects, the sensor is a conductive sensor.

In some aspects, a method of detecting a kink within a disease management device, the method comprising: passing fluid from a fluid source to a pump through a fluid flow path within the disease management device; passing fluid from the pump to a kink detector through the fluid flow path; sensing movement of a diaphragm connected to the kink detector in response to the fluid passing; measuring pressure of the fluid based on movement of the diaphragm; determining if a kink is present within the fluid flow path based on the pressure of the fluid exceeding a predetermined pressure threshold; and passing fluid from the kink detector to a cannula located at an implantation site of a patient.

In some aspects, the method of detecting a kink within a disease management device further comprising alerting a patient of the kink based on a determination of a kink presence.

In some aspects, a capacitive sensor senses movement of the diaphragm.

In some aspects, a conductive sensor senses movement of the diaphragm.

In some aspects, the conductive sensor is located within a conductive sensing circuit comprising: a conductive strip configured to measure a force of the diaphragm; a bias resistor; and a controller.

In some aspects, the conductive strip is configured to stretch as the pressure in the fluid is increased.

In some aspects, the capacitive sensor is located within a capacitive sensing circuit comprising: a bias resistor; a touchpad; a capacitor; and a controller.

In some aspects, the capacitor measures an electrical capacitance of the touchpad.

In some aspects, the electrical capacitance is configured to correspond to the movement of the diaphragm.

In some aspects, a method of detecting a kink within medication delivery system, the method comprising: passing fluid from a fluid source to a pump through a fluid flow path within the disease management device; passing fluid from the pump to a kink detector through the fluid flow path; and sensing an increase in pressure in the fluid or a lack of a flow of fluid when the flow is expected.

In some aspects, a disease management device comprising: a medical delivery pump configured to deliver a flow of medication to a patient; and a kink detection system having a sensor, the kink detection system configured to indicate an increase in pressure in the flow or a lack of the flow when the flow is expected.

### BRIEF DESCRIPTION OF THE DRAWINGS

These and other sample aspects of the disclosure will be described in the detailed description and the appended claims that follow, and in the accompanying drawings. To easily identify the discussion of any particular element or act, the most significant digit or digits in a reference number refer to the figure number in which that element is first introduced.

FIG. 1 illustrates an example disease management system that may be part of a disease management environment or used as an interleaved device.

FIG. 2 illustrates an example kink detection system.

FIG. 3A illustrates an example conductive sensing circuit that may be used in a kink detection system.

FIG. 3B illustrates an example capacitive sensing circuit that may be used in a kink detection system.

FIG. 4 illustrates example kink detection data.

### DETAILED DESCRIPTION

Disclosed herein are systems and methods for detecting one or more obstructions or kinks in a cannula or catheter configured to deliver fluid to a patient. The cannula or catheter may be part of a minimally invasive implant, such as a disease management system configured to deliver medication to a patient using a pump system. One example of such devices include an insulin pump or device that includes an insulin pump. However, other applications of the kink detections systems and methods are also possible.

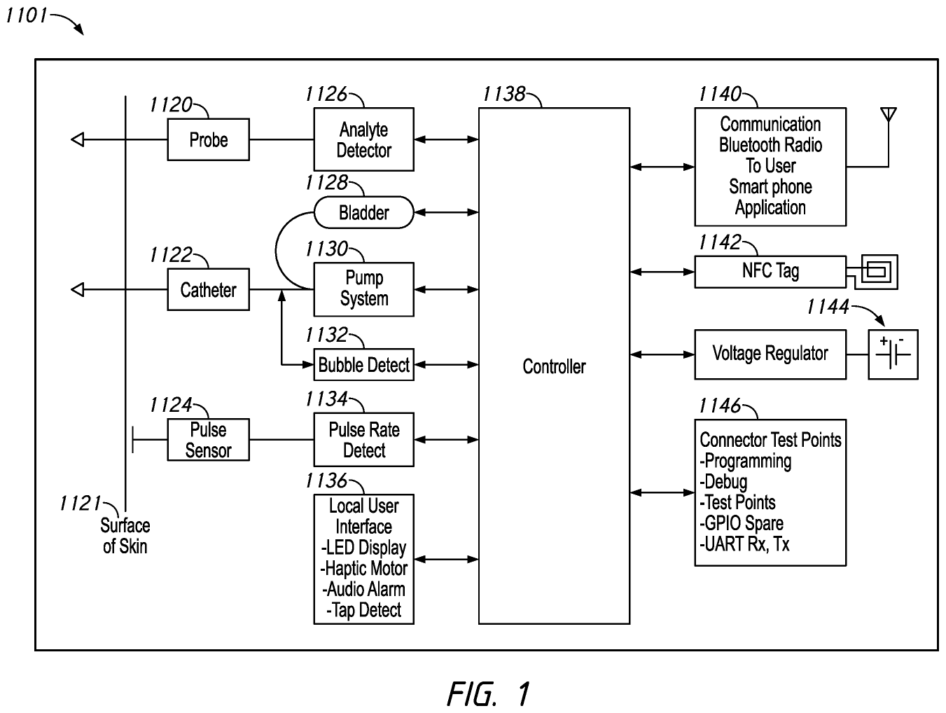

FIG. 1 shows a block diagram of an example disease management system 1101. In some examples, the disease management system 1101 may be part of a disease management environment, such as described above. A disease management system 1101 may be configured to measure one or more physiological parameters of a patient (such as pulse, skin temperature, or other values), measure one or more analytes present in the blood of a patient (such as glucose, lipids, or other analyte) and administer medication (such as insulin, glucagon, or other medication). In some examples, a disease management system 1101 may be configured to communicate with one or more hardware processors that may be external to the disease management system 1101, such as a cloud based processor or user device. A disease management system 1101 may include an NFC tag to support authentication and pairing with a user device (for example, smart phone or smart watch), bluetooth communication with additional disease management systems or devices, and bluetooth communication with a paired user device running an associated control application. To support ease of use and safe interaction with the patient, the system may incorporate user input through a tap-detecting accelerometer and provide feedback via an audio speaker, haptic vibration, and/or optical indicators.

The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 1, a controller 1138 of the disease management system 1101 may be configured to communicate and control one or more components of the disease management system 1101. The controller 1138 may include one or more hardware processors, such as a printed circuit board (PCB) or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer medication, such as insulin, by actuation of an actuated pump. The controller 1139 may record device activity and transfer the recorded data to non-volatile secure memory space. At the end of the life of a device or system, the controller can be configured to lock operation, and create a data recovery module to permit authenticated access to the recorded data if needed.

A disease management system 1101 may include an analyte sensor 1120. The analyte sensor 1120 may be configured to detect analytes in the patient's blood. For example, an analyte sensor 1120 can include a glucose sensing probe configured to pierce the surface of the skin 1121. In some examples, a disease management system 1101 may include a plurality of analyte sensors 1120 to detect one or more analytes. In some examples, an analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

A disease management system 1101 may include a medication catheter 1122. The medication catheter 1122 may be configured to administer medication, including, but not limited to insulin, to the patient. The medication catheter 1122 may receive medication from a medication bladder 1128 configured to contain medication to be administered. The medication bladder 1128 may be configured to contain medication for a prolonged period, such as 1 day, 3 days, 6 days, or more. The medication bladder 1128 may be configured to contain certain medication types, such as insulin. In some examples, a disease management system 1101 may include a plurality of medication bladders 1128 for one or more reservoirs of the same or different medications. In some examples, a disease management system 1101 may be configured to mix medications from medication bladders 1128 prior to administration to the patient. A pump system 1130 may be configured to cause medication to be administered from the bladder 1128 to the patient through the insulin catheter 1122. A pump system 1130 may include, but is not limited to, a pump, such as a nitinol wire pump or other pump configured to deliver medication to the patient from the bladder 1128 through the catheter 1122 or referred to herein as a cannula.

A disease management system 1101 may optionally include a physiological sensor. A physiological sensor may include a pulse rate sensor, such as a pulse rate sensor 1124, temperature sensor, pulse oximeter, the like or a combination thereof. In some examples, a disease management system 1101 may be configured to include a plurality of physiological sensors. The pulse rate sensor 1124 may be configured to communicate with a pulse rate detector 1134. The pulse rate detector 1134 may be configured to receive a signals of the pulse rate sensor 1124. The pulse rate detector 1134 may be configured to measure or determine and communicate a physiological value from the signal. The pulse rate detector 1134 may be configured to communicate with the controller 1138. For example, the pulse rate detector 1138 may be configured to, for example, send measured physiological values to the controller 1138 and receive control signals from the controller.

A disease management system 1101 may include one or more local user interfacing components 1136. For example, a local user interfacing component 1136 may include, but is not limited to one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, the like or a combination thereof. In some examples, a user input detector may include an inertial measurement unit, tap detector, touch display, or other component configured to accept and receive user input. In some examples, audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include one or more communication components 1140. A communication component 1140 can include, but is not limited to one or more radios configured to emit bluetooth, cellular, wi-fi, or other wireless signals. In some examples, a communication component 1140 can include a port for a wired connection. Additionally, a disease management system 1101 may include a near field communication (“NFC”) tag 1142 to facilitate in communicating with one or more hardware processors. The one or more communication components 1140 and NFC tag 1142 may be configured to communicate with the controller 1138 in order to send and/or receive information associated with the disease management system 1101. For example, a controller 1138 may communicate medication information and measured values through the one or more communication components 1140 to an external device. Additionally, the controller 1138 may receive instructions associated with measurement sampling rates, medication delivery, or other information associated with operation of the management system 1101 through the one or more communication components 1140 from one or more external devices.

A disease management system 1101 may include one or more power components 1144. The power components may include, but are not limited to one or more batteries and power management components, such as a voltage regulator. Power from the one or more power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake up mode. The wake up mode may be triggered by an NFC device and allow the system to pair with an external device with, for example, bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculations, are performed and data is communicated to an external device and/or the cloud. In another example, a disease management system 1101 may include a pump mode where the system is configured to enter a higher power mode where the pump draws power to deliver medication to the patient.

A disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, the like or a combination thereof.

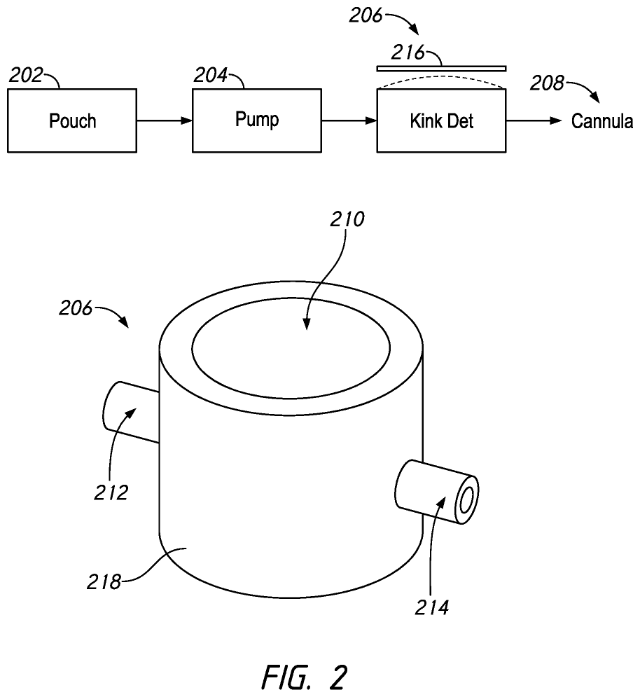

FIG. 2 illustrates an example kink detection system of a disease management system. In the illustrated example, a kink detection system may include a fluid source, such as a medication pouch 202, a pump 204, a kink detector 206, and a cannula 208. As illustrated, fluid may pass from the pouch 202 to the pump 204 to the kink detector 206 to the cannula 208. The cannula 208 may be embedded in or at an implantation site of a patient during use to deliver the fluid to the patient. The pouch 202, pump 204, and/or kink detector 206 may be configured to be contained in a single device of a disease management system. In some aspects, the pouch 202 may be a bladder 1128 of a disease management system (such as illustrated in FIG. 1). The bladder may contain the fluid that may be delivered to the patient. In some examples, the fluid may be a liquid medication. In some aspects, a pump system 1130 of the disease management system (such as illustrated in FIG. 1) may include the pump 204 and kink detector 206. The pump may be configured to initiate a flow of the fluid from the bladder through the disease management system to then be delivered to the patient.

The pump 204 may be any number of pump types, including but not limited to a muscle wire pump or another pump. Examples of pumps include, but are not limited to, those described in U.S. patent application Ser. No. 17/161,528, titled “REDUNDANT STAGGERED GLUCOSE SENSOR DISEASE MANAGEMENT SYSTEM,” filed Jan. 28, 2021, the entire disclosure of which is hereby incorporated by reference herein in its entirety. The pump 204 may be configured to transmit fluid from the pouch 202 to the kink detector 206 and subsequently the cannula 208. As described in the above referenced application, the pump can be configured to accurately and/or precisely transmit the fluid accordingly based on the dosage or amount needed by the patient.

The kink detector 206 may include a sensor 216 configured to sense movement of a diaphragm 210. The diaphragm 210 may be connected to a portion of the kink detector 206 configured to receive fluid at an inlet 212 and let fluid flow out of the portion at an outlet 214. The receiving portion 218 of the kink detector 206 may be configured to be sealed such that the path of the fluid is from the inlet 212 to the outlet 214. As pressure of the fluid increases, the diaphragm 210 may be configured to extend or move towards the sensor 216 proportionally (linearly or non-linearly) with a pressure of the fluid in the receiving portion 218 of the kink detector 206.

The sensor 216 may further be configured to indicate a lack of flow of the fluid. In some aspects, the sensor 216 may expect a flow of fluid when the sensor 216 does not sense movement of the diaphragm. In some aspects, a lack of flow of the fluid can cause the diaphragm 210 to move or increase in size. This can cause an increase in pressure within the fluid. An increase in pressure within the fluid can correlate to a lack of flow of the fluid when flow may be expected. For example, the sensor 216 may expect a flow of the fluid, but the sensor 216 may instead sense a movement of the diaphragm 210. As explained above, the diaphragm 210 may extend or move in a manner that may cause the sensor 216 sense a movement of the diaphragm 210. This can cause the sensor 216 to indicate there is a lack of flow of the fluid.

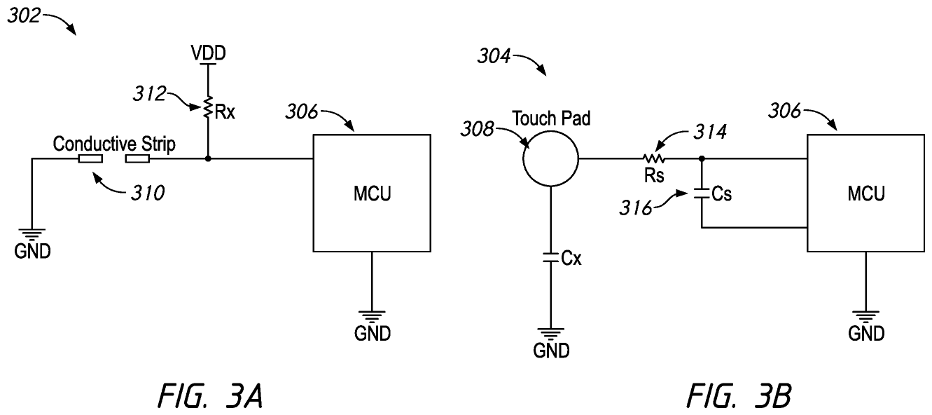

FIG. 3A and FIG. 3B illustrate example circuit diagrams of example variations of the kink detector 206. As illustrated in FIG. 3A, a kink detector 206 may include a conductive sensing circuit 302. The conductive sensing circuit 302 can include, but is not limited to a bias resistor 312, a conductive strip 310 and a microcontroller circuit 306. A conductive sensing circuit 302 can include a conductive strip 310 or other conductive flexible material. The conductive strip 310 or other conductive flexible material may be configured to measure a stretch force of the diaphragm 210. The more the conductive strip 310 stretches, the more the resistance of the conductive strip 310 increases. Based on this change in resistance, a microcontroller circuit 306 can determine a pressure of the fluid in the system. If there is a kink or other obstruction in the cannula or elsewhere in the fluid flow path, there may be an increased pressure in the system. This increased pressure can be identified and if it exceeds a threshold, the disease management system can alert a user of the potential kink or obstruction. The microcontroller circuit 306 can further indicate a lack of flow within the system. In some aspects, microcontroller circuit 306 may expect a flow of fluid when the microcontroller circuit 306 determines a pressure of the fluid in the system that may equate to no kink or an unobstructed fluid flow path or cannula. In some aspects, a lack of flow of the fluid can cause the microcontroller circuit 306 to measure or determine an increase in pressure of the fluid in the system. As described above, this can correlate to a kink or obstruction in the fluid flow path, that may otherwise not be expected to be present. An increase in pressure within the fluid can correlate to a lack of flow of the fluid when flow may be expected. For example, the microcontroller circuit 306 may expect a flow of the fluid, but the microcontroller circuit 216 may instead determine an increase in pressure of the fluid in the system. As explained above, an increase in pressure may correlate to the presence of a kink or obstruction within the fluid flow path. This can cause the microcontroller circuit 306 to indicate there is a lack of flow of the fluid.

FIG. 3B illustrates an example kink detector 206 that include a capacitive sensing circuit 304. The capacitive sensing circuit 304 can include, but is not limited to a bias resistor 314, a touchpad 308, a capacitor 316 and a microcontroller circuit 306. A capacitive sensing circuit 304 can include a touchpad 308 or other capacitive sensor. An electrical capacitance of the touchpad 308 may change based on the movement of the diaphragm 210. Based on this change in capacitance, a microcontroller circuit 306 may determine the pressure of the fluid in the system. In a similar manner as referenced above, if there is a kink or other obstruction in the cannula or elsewhere in the fluid flow path, the disease management system can alert a user of the potential kink or obstruction. Similarly, the disease management system can alert a user of the lack of flow within the fluid flow path of the system. In some examples, the system may alert the user after the pressure reaches a threshold or a predetermined threshold.

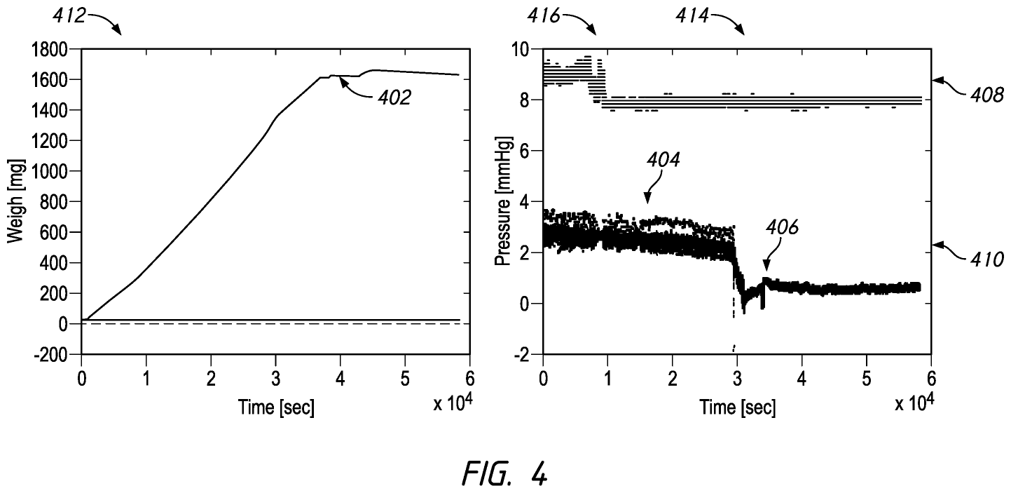

FIG. 4 illustrates example pump test result. Graph 412 illustrates an example of the amount of fluid dispensed over a period of time. In the illustrated graph 412, an empty syringe event 402 or empty cannula event is shown at point 402. Graph 414 illustrates an example of back pressure 408 and inlet pressure 410 over the same period. This back pressure 408 may be measured by the kink detector 206. An empty syringe event 402 is shown at point 406 as a dip in inlet pressure 410. A gradual fluid reduction 404 inside the syringe or cannula is shown in the slow reduction in inlet pressure 410 around point 404 from 0 to 3×10\hat{ }4 seconds. As illustrated, an area 416 of the graph of back pressure 408 may be slightly elevated during a potential kink or obstruction. In some aspects, the larger the kink, the greater the pressure measured.

### Terminology

While the above description has pointed out novel features of the invention as applied to various embodiments, the skilled person will understand that various omissions, substitutions, and changes in the form and details of the device or process illustrated may be made without departing from the scope of the invention. Therefore, the scope of the invention is defined by the appended claims rather than by the foregoing description. All variations coming within the meaning and range of equivalency of the claims are embraced within their scope.

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics can be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Embodiments of the disclosed systems and methods can be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above.

What is claimed is:

1. A disease management device comprising:
a medical delivery pump configured to deliver a medication from a medication pouch to a patient via a cannula implanted in a patient; and
a kink detection system configured to measure an obstruction in the cannula, the kink detection system comprising:
a fluid receiving portion configured to receive fluid from the medical delivery pump;
a diaphragm coupled to the fluid receiving portion, the diaphragm configured to deform in correspondence with a change in pressure in the fluid received;
a sensor configured to measure an extent of deformation of the diaphragm;
one or more hardware processors configured to:
determine a pressure in the fluid based on the extent of deformation;
determine if a change in the pressure in the fluid passes a threshold change; and
output an alert to the patient if the change in pressure passes the threshold change; and
a fluid outlet configured to deliver the fluid to the patient.

2. The disease management device of claim 1, wherein the sensor comprises a capacitive sensor.

3. The disease management device of claim 1, wherein the sensor comprises a conductive sensor.

4. The disease management device of claim 2, wherein the capacitive sensor is located with in a capacitive sensing circuit, the capacitive sensing circuit comprising:
a bias resistor;
a touchpad;
a capacitor; and
a controller.

5. The disease management device of claim 4, wherein an electrical capacitance of the touchpad is configured to correspond to movement of the diaphragm.

6. The disease management device of claim 3, wherein the conductive sensor is located within a conductive sensing circuit, the conductive sensing circuit comprising:
a bias resistor;
a conductive strip; and
a controller.

7. The disease management device of claim 6, wherein the conductive strip is configured to stretch as the pressure in the fluid is increased.

8. The disease management device of claim 1, wherein the one or more hardware processors are further configured to communicate with peripheral devices via bluetooth.

9. A detection system configured to detect an obstruction within a fluid flow path, the detection system comprising:
a fluid receiving portion configured to receive fluid from a delivery pump;
a diaphragm coupled to the fluid receiving portion, the diaphragm configured to deform in correspondence with a change in pressure in the fluid received;
a sensor configured to measure an extent of deformation of the diaphragm; and
one or more hardware processors configured to:
determine a pressure in the fluid based on the extent of deformation;
determine if a change in the pressure in the fluid passes a predetermined pressure threshold; and
output an alert to a user if the change in pressure passes the predetermined pressure threshold.

10. The detection system of claim 9, wherein the sensor is a capacitive sensor.

11. The detection system of claim 9, wherein the sensor is a conductive sensor.

12. A method of detecting a kink within a disease management device, the method comprising:
passing fluid from a fluid source to a pump through a fluid flow path within the disease management device;
passing fluid from the pump to a kink detector through the fluid flow path;
sensing movement of a diaphragm connected to the kink detector in response to the fluid passing;
measuring pressure of the fluid based on movement of the diaphragm;
determining if a kink is present within the fluid flow path based on the pressure of the fluid exceeding a predetermined pressure threshold; and
passing fluid from the kink detector to a cannula located at an implantation site of a patient.

13. The method of claim 12 further comprising alerting a patient of the kink based on a determination of a kink presence.

14. The method of claim 12, wherein a capacitive sensor senses movement of the diaphragm.

15. The method of claim 12, wherein a conductive sensor senses movement of the diaphragm.

16. The method of claim 15, wherein the conductive sensor is located within a conductive sensing circuit comprising:
a conductive strip configured to measure a force of the diaphragm;
a bias resistor; and
a controller.

17. The method of claim 16, wherein the conductive strip is configured to stretch as the pressure in the fluid is increased.

18. The method of claim 14, wherein the capacitive sensor is located within a capacitive sensing circuit comprising:
a bias resistor;
a touchpad;
a capacitor; and
a controller.

19. The method of claim 18, wherein the capacitor measures an electrical capacitance of the touchpad.

20. The method of claim 19, wherein the electrical capacitance is configured to correspond to the movement of the diaphragm.

21. A method of detecting a kink within medication delivery system, the method comprising:
passing fluid from a fluid source to a pump through a fluid flow path within the disease management device;
passing fluid from the pump to a kink detector through the fluid flow path; and
sensing an increase in pressure in the fluid or a lack of a flow of fluid when the flow is expected.

22. A disease management device comprising:
a medical delivery pump configured to deliver a flow of medication to a patient; and
a kink detection system having a sensor, the kink detection system configured to indicate an increase in pressure in the flow or a lack of the flow when the flow is expected.
